(19) United States
(12) Patent Application Publication
(10) Pub. No.: US 2023/0094832 A1
(43) Pub. Date: Mar. 30, 2023

(54) ANTIBODY-DRUG CONJUGATE COMPRISING IMMUNE CHECKPOINT INHIBITOR AND EXOSOME SECRETION INHIBITOR, AND PHARMACEUTICAL COMPOSITION COMPRISING SAME

(71) Applicants: Jae Hyung PARK, Suwon-si (KR); Jung Min SHIN, Hwaseong-si (KR); So Young SON, Gunpo-si (KR)

(72) Inventors: Jae Hyung PARK, Suwon-si (KR); Jung Min SHIN, Hwaseong-si (KR); So Young SON, Gunpo-si (KR)

(73) Assignee: Research & Business Foundation Sungkyunkwan University, Suwon-si (KR)

(21) Appl. No.: 17/796,413

(22) PCT Filed: Jan. 27, 2021
(86) PCT No.: PCT/KR2021/001063

§ 371 Date: Jul. 29, 2022

(30) Foreign Application Priority Data
Jan. 30, 2020 (KR) 10-2020-0011213
Jan. 26, 2021 (KR) 10-2021-0010554

(51) Int. Cl. A61K 47/68; C07K 16/28; A61K 47/65; A61K 31/513; A61P 35/00

(52) U.S. Cl. CPC A61K 47/68; C07K 16/28; A61K 47/65; A61K 31/513; A61P 35/00

(57) ABSTRACT

The present invention relates to an antibody-drug conjugate comprising an exosome secretion inhibitor conjugated to an antibody for inhibiting immune checkpoints, and the use thereof for treating cancer. An antibody-drug conjugate according to the present invention is maintained, in normal tissue, in a form in which a drug is conjugated to an antibody, and releases the drug upon reaching a cancer microenvironment, thereby inhibiting the secretion of cancer exosomes that cause an immunosuppressive mechanism. Thus the antibody-drug conjugate exhibits high therapeutic efficacy and can remarkably increase the objective response rate to an immune checkpoint inhibitor.

### TECHNICAL FIELD

The present invention relates to an antibody-drug conjugate comprising an exosome secretion inhibitor conjugated to an antibody for inhibiting immune checkpoints, and the use thereof for treating cancer.

This application claims priority to and the benefit of Korean Patent Application Nos. 10-2020-0011213 and 10-2021-0010554 filed in the Korean Intellectual Property Office on Jan. 30, 2020 and Jan. 26, 2021, respectively, and all the contents disclosed in the specification and drawings of those applications are incorporated in this application.

### BACKGROUND ART

Resection of tumor tissue through surgery is the most preferred as a general method for treating cancer. However, recently, non-invasive cancer treatment methods due to problems such as sequelae of invasive treatment methods including surgery have been attracting attention, and active research has been conducted. In particular, anti-cancer treatment using an antibody for inhibiting immune checkpoints has been attracting attention as an efficient cancer treatment method.

Anti-cancer immunotherapy utilizing an immune checkpoint inhibitor is a method of activating cytotoxic T cells and the like and inducing the apoptosis of cancer cells by suppressing immune checkpoint interactions such as programmed cell death 1 (PD-1)/PD-1 ligand 1 (PD-L1) which inhibit immune responses in the human body using the corresponding antibody (anti-PD-1 antibody, anti-PD-L1 antibody, and the like), and this method has been widely utilized in clinical settings due to excellent therapeutic efficacy compared to existing treatment methods.

However, treatment using an antibody for inhibiting immune checkpoints has been found to be ineffective in a significant number of patients (>70%), and this is because cancer has an active immunosuppressive mechanism such as construction of a cancer microenvironment that facilitates immunosuppression by secreting cancer exosomes. Although studies have been attempted to improve the efficiency of anti-cancer immunotherapy through combination therapy with existing cancer therapies such as chemotherapy in order to improve the limitations of such anti-cancer immunotherapy, there is a need for a fundamental solution that seeks to overcome the mechanism of immunosuppression and immune escape of cancer.

### DISCLOSURE

### Technical Problem

Thus, the present inventors prepared a novel antibody-drug conjugate (ADC) in a form in which a drug is conjugated to an antibody for inhibiting immune checkpoints through a cleavable linker designed such that an exosome secretion inhibitor is cleaved in a cancer microenvironment, and confirmed that the prepared antibody-drug conjugate effectively inhibits the secretion of cancer exosomes which cause the immunosuppressive mechanism of cancer cells by releasing the exosome secretion inhibitor, which is a drug, and thus, can remarkably enhance an anti-cancer effect caused by the antibody for inhibiting immune checkpoints, thereby completing the present invention.

Therefore, an object of the present invention is to provide an antibody-drug conjugate including: an antibody which is an immune checkpoint inhibitor; and an exosome secretion inhibitor conjugated to the antibody through a linker, or a pharmaceutically acceptable salt thereof.

Another object of the present invention is to provide a pharmaceutical composition for preventing or treating cancer, including the antibody-drug conjugate or a pharmaceutically acceptable salt thereof as an active ingredient.

However, the technical problems which the present invention intends to solve are not limited to the technical problems which have been mentioned above, and other technical problems which have not been mentioned will be apparently understood by a person with ordinary skill in the art to which the present invention pertains from the following description.

### Technical Solution

To achieve the objects of the present invention, the present invention provides an antibody-drug conjugate including: an antibody which is an immune checkpoint inhibitor; and an exosome secretion inhibitor conjugated to the antibody through a linker, or a pharmaceutically acceptable salt thereof.

Further, the present invention provides a pharmaceutical composition for preventing or treating cancer, including the antibody-drug conjugate or a pharmaceutically acceptable salt thereof as an active ingredient.

Furthermore, the present invention provides a method for preventing or treating cancer, the method including: administering the antibody-drug conjugate or a pharmaceutically acceptable salt thereof to a subject in need thereof.

In addition, the present invention provides a use of the antibody-drug conjugate or a pharmaceutically acceptable salt thereof for the prevention, amelioration or treatment of cancer.

Furthermore, the present invention provides a use of the antibody-drug conjugate or a pharmaceutically acceptable salt thereof for the preparation of a preparation for preventing or treating cancer.

In an exemplary embodiment of the present invention, the antibody, which is an immune checkpoint inhibitor may be an antibody that specifically binds to programmed cell death 1 (PD-1) or PD-1 ligand 1 (PD-L1), but is not limited thereto.

In another exemplary embodiment of the present invention, the antibody that specifically binds to PD-1 may be pembrolizumab, nivolumab or cemiplimab, but is not limited thereto.

In still another exemplary embodiment of the present invention, the antibody that specifically binds to PD-L1 may be atezolizumab, avelumab or durvalumab, but is not limited thereto.

In yet another exemplary embodiment of the present invention, the antibody, which is an immune checkpoint inhibitor may be an antibody that specifically binds to cytotoxic T-lymphocyte-associated protein 4 (CTLA4) or lymphocyte activation gene-3 (LAG-3), but is not limited thereto.

In yet another exemplary embodiment of the present invention, the antibody that specifically binds to CTLA4 may be ipilimumab, but is not limited thereto. In yet another exemplary embodiment of the present invention, the exosome secretion inhibitor may be selected from the group consisting of Manumycin A, GW4869, cannabidiol and an endothelin receptor antagonist, but is not limited thereto.

In yet another exemplary embodiment of the present invention, the endothelin receptor antagonist may be selected from the group consisting of ambrisentan, sulfisoxazole, BQ-123, BQ-788, zibotentan, sitaxentan, atrasentan, bosentan, macitentan, tezosentan and A192621, but is not limited thereto.

In yet another exemplary embodiment of the present invention, the linker is a cleavable linker which is cleaved in a cancer microenvironment, and the exosome secretion inhibitor may be released by cleavage of the linker, but the linker is not limited thereto.

In yet another exemplary embodiment of the present invention, the linker is a cleavable linker which is cleaved by a protease, and the exosome secretion inhibitor may be released by cleavage of the linker, but the linker is not limited thereto.

In yet another exemplary embodiment of the present invention, the protease may be selected from the group consisting of Cathepsin B, Cathepsin K, a matrix metalloproteinase (MMP) and urokinase, but is not limited thereto.

In yet another exemplary embodiment of the present invention, the linker is a cleavable linker which is cleaved by acidity or reactive oxygen species of a cancer microenvironment, and the exosome secretion inhibitor may be released by cleavage of the linker, but the linker is not limited thereto.

In yet another exemplary embodiment of the present invention, the linker may be a peptide linker, but is not limited thereto.

In yet another exemplary embodiment of the present invention, the peptide linker may be a cleavable linker which is cleaved by a protease, but is not limited thereto.

In yet another exemplary embodiment of the present invention, the peptide linker may be a valine-citrulline linker, but is not limited thereto.

In yet another exemplary embodiment of the present invention, the cancer may be a cancer selected from the group consisting of lung cancer, gastric cancer, gliomas, liver cancer, melanoma, renal cancer, urothelial carcinoma, head and neck cancer, Merkel-cell carcinoma, prostate cancer, hematologic malignancy, breast cancer, colorectal cancer, colon cancer, rectal cancer, pancreatic cancer, brain cancer, ovarian cancer, bladder cancer, bronchial cancer, skin cancer, cervical cancer, endometrial cancer, esophageal cancer, thyroid cancer, bone cancer and a combination thereof, but is not limited thereto.

### Advantageous Effects

The present invention relates to an immune checkpoint inhibitor-based cancer therapy capable of overcoming a cancer microenvironment that facilitates immunosuppression along with anti-cancer immunotherapy. Specifically, it is known that cancer exosomes are secreted by cancer cells and contribute to making cancer tissue an immunosuppressive microenvironment. Further, recently, it has been revealed that PD-L1 of exosomes is known as the main cause of suppressing the function of T cells through the bloodstream and inhibits the efficacy of immune checkpoint inhibitors. Accordingly, a limitation that when the therapeutic efficacy of an immune checkpoint inhibitor is limited, an objective response rate is also reduced has been revealed.

An antibody-drug conjugate according to the present invention is maintained, in normal tissue, in a form in which a drug is conjugated to an antibody, and releases the drug upon reaching a cancer microenvironment, thereby inhibiting the secretion of cancer exosomes that cause an immunosuppressive mechanism. Thus the antibody-drug conjugate exhibits high therapeutic efficacy and can remarkably increase the objective response rate to an immune checkpoint inhibitor.

### DESCRIPTION OF DRAWINGS

FIG. 1 is a view illustrating the ¹H-NMR results of a linker-drug conjugate (VC-AMB) according to an exemplary embodiment of the present invention.

FIG. 2 is a view illustrating exemplary components of an antibody-drug conjugate according to an exemplary embodiment of the present invention.

FIG. 3 is a view illustrating the results of measuring absorbance for confirming the drug-to-antibody ratio (DAR) of Ab-VC-AMB (ADC) which is an antibody-drug conjugate according to an exemplary embodiment of the present invention.

FIG. 4 is a view illustrating the cytotoxicity of Ab, AMB, AMB+Ab, and Ab-VC-AMB for a melanoma cell line B16F10.

FIG. 5 is a view illustrating the results of intravenously injecting Ab-VC-AMB (ADC), saline, an antibody (Ab) or a drug (AMB drug) into a cancer animal model, and then observing tumor volumes among the administration material groups for 11 days. A red arrow indicates the date of administration of each material.

FIG. 6 is a view illustrating changes in tumor volume for each individual animal in a cancer animal model into which each material (ADC, an AMB drug, an antibody, and saline) was intravenously administered.

FIG. 7 is a view illustrating changes in body weight of the cancer animal model into which each material was administered.

FIG. 8 is a view illustrating the histopathological evaluation results according to the administration of each material (ADC, an AMB drug, an antibody, and saline) obtained by performing H&E staining on the main organs and cancer tissues of the cancer animal model.

FIG. 9 is a view illustrating the results of isolating exosomes in plasma from a cancer animal model and measuring exosomal total protein amounts after completing an anti-cancer effect (change in tumor volume) experiment.

FIG. 10 is a view illustrating the results of isolating exosomes in plasma from a cancer animal model and measuring exosomal PD-L1 amounts after completing an anti-cancer effect (change in tumor volume) experiment.

### MODES OF THE INVENTION

Hereinafter, the present invention will be described in detail.

The present invention provides an antibody-drug conjugate including: an antibody which is an immune checkpoint inhibitor; and an exosome secretion inhibitor conjugated to the antibody through a linker, or a pharmaceutically acceptable salt thereof.

As used herein, the term “immune checkpoint inhibitor” refers to a material that totally or partially inhibits, interferes with or regulates one or more immune checkpoint proteins. Immune checkpoint proteins regulate the activation or function of T cells. A plurality of immune checkpoint proteins such as PD-1, PD-L1 and CTLA-4 have been publicly known (Nature Reviews Cancer 12: 252-264, 2012). These proteins are involved in the co-stimulatory or inhibitory interactions of T cell responses. Immune checkpoint inhibitors include antibodies, and may originate from antibodies.

In the present invention, the antibody, which is an immune checkpoint inhibitor may be an antibody that specifically binds to programmed cell death 1 (PD-1) or PD-1 ligand 1 (PD-L1). In an exemplary embodiment, the antibody that specifically binds to PD-1 may be an antibody selected from the group consisting of pembrolizumab, nivolumab and cemiplimab. Further, in an exemplary embodiment, the antibody that specifically binds to PD-L1 may be an antibody selected from the group consisting of atezolizumab, avelumab and durvalumab.

In the present invention, the antibody, which is an immune checkpoint inhibitor may be an antibody that specifically binds to cytotoxic T-lymphocyte-associated protein 4 (CTLA4) or lymphocyte activation gene-3 (LAG-3). In an exemplary embodiment, the antibody that specifically binds to CTLA4 may be ipilimumab.

As used herein, the term “antibody” refers to a material that specifically binds to immune checkpoint proteins such as PD-1, PD-L1 and CTLA4 to exhibit immune checkpoint inhibitory activity. In the antibody-drug conjugate of the invention, the scope of the antibody conjugated to a drug includes not only the intact form of the antibody, but also the antigen binding site of the antibody molecule.

An intact antibody is a structure having two full-length light chains and two full-length heavy chains, and each light chain is linked to each heavy chain by a disulfide bond. A heavy chain constant region has gamma (γ), mu (μ), alpha (α), delta (δ) and epsilon (ε) types, and has gamma 1 (γ1), gamma 2 (γ2), gamma 3 (γ3), gamma 4 (γ4), alpha 1 (α1) and alpha 2 (α2) as a subclass. A light chain constant region has kappa (κ) and lambda (λ) types.

The antigen binding site of the antibody molecule refers to a fragment having an antigen binding function, and includes Fab, F(ab′), F(ab′)₂, and Fv, and the like. Among the antibody fragments, Fab has one antigen binding site with a structure having variable regions of a light chain and a heavy chain and a constant region of the light chain and a first constant region (CH_{H1}) of the heavy chain. Fab′ differs from Fab in that Fab′ has a hinge region including one or more cysteine residues at the C-terminus of the heavy chain CH_{H1} domain. The F(ab′)₂ antibody is produced while the cysteine residue of the hinge region of Fab′ forms a disulfide bond. Fv is the smallest antibody fragment that has only a heavy chain variable region and a light chain variable region, and recombinant techniques for producing Fv fragments are disclosed in PCT International Publication Nos. WO88/10649, WO 88/106630, WO 88/07085, WO 88/07086, WO 88/09344, and the like.

The antibody of the present invention includes a monoclonal antibody, a multispecific antibody, a human antibody, a humanized antibody, a chimeric antibody, and the like, but is not limited thereto.

As used herein, the term “exosome secretion inhibitor” refers to a drug that blocks or inhibits exosome production (biogenesis), exosome secretion, or both in cancer cells.

In the present invention, the exosome secretion inhibitor may be selected from the group consisting of Manumycin A, GW4869, cannabidiol and an endothelin receptor antagonist.

As used herein, the term “endothelin receptor antagonist” refers to a drug that acts on an endothelin receptor molecule in vivo to suppress or inhibit the function thereof.

In the present invention, the endothelin receptor antagonist may be selected from the group consisting of ambrisentan, sulfisoxazole, BQ-123, BQ-788, zibotentan, sitaxentan, atrasentan, bosentan, macitentan, tezosentan and A192621.

In the present invention, the exosome secretion inhibitor may suppress exosome production and/or secretion in cancer. Cancer produces and secretes exosomes, and the secreted exosomes contain materials such as proteins (for example, PD-L1) that are needed to suppress the anti-cancer immune response. The secretion of such cancer exosomes suppresses the anti-cancer immune response, and the therapeutic effect by the immune checkpoint inhibitor deteriorates. Further, PD-L1 of exosomes is known as the main cause of suppressing the function of T cells through the bloodstream. In an exemplary embodiment of the present invention, it was confirmed that when the antibody-drug conjugate of the present invention reaches a cancer microenvironment, as a linker is cleaved, an exosome secretion inhibitor (ambrisentan), which is a drug, is released, and the secretion of cancer exosomes is inhibited by the released drug (see Example 4).

As used herein, the term “linker” refers to a compound that is a component of an antibody-drug conjugate and covalently binds an exosome secretion inhibitor to an antibody for inhibiting immune checkpoints.

In the present invention, the linker may be designed as a cleavable linker which is cleaved in a cancer microenvironment. The cleavage of the linker releases the exosome secretion inhibitor from the antibody-drug conjugate.

The cleavable linker may be a linker designed to be cleaved in response to characteristic elements of the cancer microenvironment (pH, ROS, enzymes, hypoxia, etc.), which are distinguished from normal tissues. In an exemplary embodiment, the linker may be a cleavable linker which is cleaved by a protease. For example, the protease may be a lysosomal enzyme, for example, Cathepsin B, which is an enzyme overexpressed in a cancer microenvironment. In another exemplary embodiment, the linker may be a cleavable linker which is cleaved by acidity or reactive oxygen species of a cancer microenvironment.

In the present invention, the protease may be an enzyme selected from the group consisting of Cathepsin B, Cathepsin K, a matrix metalloproteinase (MMP) and urokinase.

In the present invention, the linker may be a peptide linker. Peptides that are constituents of the peptide linker may include 20 major amino acids and minor amino acids well known in the field of biochemistry, for example, two or more amino acid residues, including citrulline. The amino acid residue includes all stereoisomers and may be in a D or L steric configuration. For example, the peptide may be an amino acid unit including 2 to 12 amino acid residues independently selected from glycine, alanine, phenylalanine, lysine, arginine, valine and citrulline.

In an exemplary embodiment, the amino acid unit allows cleavage of the linker by a protease, promoting the release of an exome secretion inhibitor from an antibody-drug conjugate upon exposure to an intracellular proteolytic enzyme (for example, a lysosomal enzyme). For example, such an amino acid unit may include dipeptides (valine-citrulline, alanine-phenylalanine, phenylalanine-lysine, N-methyl-valine-citrulline, and the like), tripeptides (glycine-valine-citrulline, valine-citrulline-phenylalanine, glycine-glycine-glycine, and the like), tetrapeptides, pentapeptides and hexapeptides.

Peptide linkers may be prepared by forming a peptide bond between two or more amino acids and/or peptide fragments. The peptide bond may be prepared, for example, by a liquid synthesis method well known in the field of peptide chemistry (E. Schroder and K. Lubke (1965) “The Peptides”, volume 1, pp 76-136, Academic Press). Amino acid units may be designed and optimized in consideration of enzymatic cleavage by a specific enzyme, for example, a tumor-related protease, Cathepsin B, C, and D or a plasmin protease.

In the present invention, the peptide linker may be a valine-citrulline linker.

The linker of the invention may include a spacer moiety for binding the linker to an antibody. For example, the linker may include a reactive moiety having an electrophilic group that is reactive to a nucleophilic group on an antibody as a spacer moiety. The electrophilic group on the linker provides a convenient linker attachment site for the antibody. A useful nucleophilic group on an antibody includes, for example, sulfhydryl, a hydroxyl group and an amino group. A heteroatom of the nucleophilic group of the antibody is reactive to the electrophilic group on the linker and forms a covalent bond to the linker. A useful electrophilic group of the linker includes, for example, maleimide (for example, maleimidocaproyl) and a haloacetamide group.

Further, the linker may include a reactive moiety having a nucleophilic group that is reactive to an electrophilic group on an antibody as a spacer moiety. The nucleophilic group on the linker provide a convenient attachment site for the linker. A useful electrophilic group on an antibody includes, for example, aldehyde, a ketone carbonyl group and a carboxylic acid group. A heteroatom of the nucleophilic group of the linker may react with the electrophilic group on the antibody and may form a covalent bond to the antibody. A nucleophilic group of the linker includes, for example, hydrazide, oxime, amino, hydrazine, thiosemicarbazone, hydrazine carboxylate and arylhydrazide. The nucleophilic group on the linker provides a convenient attachment site for the linker.

Additionally, the linker of the present invention may include a self-immolative moiety (for example, p-aminobenzyl alcohol (PABA), p-aminobenzyloxycarbonyl (PABC), PAB-OH, and the like).

In the present invention, the antibody-drug conjugate may have the following Structural Formula 1, but is not limited thereto.

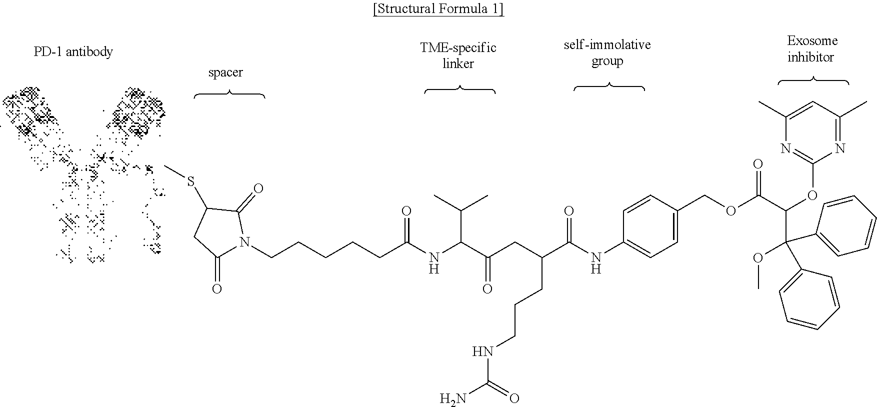 [SMILES: COC(c1ccccc1)(c1ccccc1)C(Oc1nc(C)cc(C)n1)C(=O)OCc1ccc(NC(=O)C(CCCNC(N)=O)CC(=O)C(NC(=O)CCCCCN2C(=O)CC(SC)C2=O)C(C)C)cc1]

As used herein, the term “cancer” is a disease related to the regulation of cell death, and refers to a disease caused by hyperproliferation of cells when the balance for normal cell apoptosis is broken. In some cases, such abnormal hyperproliferative cells may invade surrounding tissues and organs to form a mass and cause destruction or deformation of the normal structures in the body, and these conditions are collectively referred to as cancer.

In general, a tumor means a mass abnormally grown by autonomous overgrowth of body tissues, and may be classified into a benign tumor and a malignant tumor. A malignant tumor has a very rapid growth rate relative to a benign tumor, and causes metastasis when invading surrounding tissues, thus becoming life-threatening. Such malignant tumor is typically referred to as cancer.

Cancers that can be prevented or treated by the composition of the present invention include hematologic malignancy, colorectal cancer, brain cancer, gliomas, gastric cancer, lung cancer, cervical cancer, colon cancer, rectal cancer, throat cancer, lymphangiosarcoma, endometrial cancer, ovarian cancer, esophageal cancer, breast cancer, pancreatic cancer, prostate cancer, renal cancer, liver cancer, Merkel-cell carcinoma, cholangiocarcinoma, choriocarcinoma, testicular tumors, Wilms' tumors, Ewing's tumor, bladder cancer, angiosarcoma, papillary carcinoma, mammary line sarcoma, bronchial cancer, melanoma, leiomyoma, urothelial carcinoma, head and neck cancer, rhabdomyoma, neuroblastoma, retinoblastoma, hemangioblastoma, bone cancer, fibrosarcoma and leukemia.

In the present invention, the cancer may be selected from the group consisting of lung cancer, gastric cancer, gliomas, liver cancer, melanoma, renal cancer, urothelial carcinoma, head and neck cancer, Merkel-cell carcinoma, prostate cancer, hematologic malignancy, breast cancer, colorectal cancer, colon cancer, rectal cancer, pancreatic cancer, brain cancer, ovarian cancer, bladder cancer, bronchial cancer, skin cancer, cervical cancer, endometrial cancer, esophageal cancer, thyroid cancer, bone cancer and a combination thereof.

The present invention may also include an active ingredient in the form of a pharmaceutically acceptable salt. In the present invention, the term “pharmaceutically acceptable salt” includes a salt derived from a pharmaceutically acceptable inorganic acid, organic acid, or base.

Examples of a suitable acid include hydrochloric acid, bromic acid, sulfuric acid, nitric acid, perchloric acid, fumaric acid, maleic acid, phosphoric acid, glycolic acid, lactic acid, salicylic acid, succinic acid, toluene-p-sulfonic acid, tartaric acid, acetic acid, citric acid, methanesulfonic acid, formic acid, benzoic acid, malonic acid, gluconic acid, naphthalene-2-sulfonic acid, benzenesulfonic acid, and the like. An acid addition salt may be prepared by a typical method, for example, dissolving a compound in an excessive amount of an aqueous acid solution and precipitating the salt using a water-miscible organic solvent such as methanol, ethanol, acetone or acetonitrile. In addition, the acid addition salt may be prepared by heating the same molar amount of compound and an acid or alcohol in water, subsequently evaporating the mixture to dry the mixture, or suction-filtering the precipitated salt.

A salt derived from a suitable base may include an alkali metal such as sodium and potassium, an alkaline earth metal such as magnesium, ammonium and the like but is not limited thereto. An alkali metal or alkaline earth metal salt may be obtained by, for example, dissolving the compound in an excessive amount of alkali metal hydroxide or alkaline earth metal hydroxide solution, filtering a non-soluble compound salt, evaporating the filtrate, and drying the resulting product. In this case, it is particularly suitable to prepare a sodium, potassium or calcium salt as the metal salt from the pharmaceutical perspective, and the corresponding silver salt may also be obtained by reacting an alkali metal or alkaline earth metal salt with a suitable silver salt (for example, silver nitrate).

The content of the antibody-drug conjugate in the composition of the present invention can be appropriately adjusted according to the symptoms of the disease, the degree of progression of the symptoms, the condition of the patient, and the like, and may be, for example, 0.0001 to 99.9 wt %, or 0.001 to 50 wt %, but is not limited thereto. The content ratio is a value based on a dry amount from which the solvent is removed.

The total effective amount of the antibody-drug conjugate of the present invention may be administered in a single dose or may be administered by a fractionated treatment protocol, in which multiple doses are administered over a long period of time. The pharmaceutical composition of the present invention may vary in the content of the active ingredient depending on the extent and/or purpose of the disease, but may be administered repeatedly several times a day, typically in an effective dose of 0.01 μg to 10000 mg, preferably 0.1 μg to 1000 mg per single dose. However, for the dose of the pharmaceutical composition, an effective dose for a patient is determined in consideration of not only the formulation method, route of administration and number of times of treatment, but also various factors such the patient's age, body weight, health condition, and sex, severity of disease, diet and excretion rate, so that a person with ordinary skill in the art will be able to determine an appropriate effective dose of the composition of the present invention in consideration of these points. The pharmaceutical composition according to the present invention is not particularly limited in its dosage form, route of administration and method of administration as long as it exhibits the effects of the present invention.

The pharmaceutical composition of the present invention may further include an appropriate carrier, an appropriate excipient, and an appropriate diluent, which are typically used to prepare a pharmaceutical composition. The excipient may be, for example, one or more selected from the group consisting of a diluent, a binder, a disintegrant, a lubricant, an adsorbent, a moisturizer, a film-coating material, and a controlled release additive.

The pharmaceutical composition according to the present invention may be used by being formulated into the form of an external preparation such as a powder, a granule, a sustained-release granule, an enteric granule, a liquid, a collyrium, an elixir, an emulsion, a suspension, a spirit, a troche, aromatic water, a limonade, a tablet, a sustained-release tablet, an enteric tablet, a sublingual tablet, a hard capsule, a soft capsule, a sustained-release capsule, an enteric capsule, a pill, a tincture, a soft extract agent, a dry extract agent, a fluid extract agent, an injection, a capsule, a perfusate, a plaster, a lotion, a paste, a spray, an inhalant, a patch, a sterilized injection solution, or an aerosol, and the external preparation may have a formulation such as a cream, a gel, a patch, a spray, an ointment, a plaster, a lotion, a liniment, a paste or a cataplasma.

Examples of a carrier, an excipient or a diluent which may be included in the composition according to the present invention include lactose, dextrose, sucrose, an oligosaccharide, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and mineral oil.

When the pharmaceutical composition is prepared, the pharmaceutical composition is prepared using a diluent or excipient, such as a filler, an extender, a binder, a wetting agent, a disintegrant, and a surfactant, which are commonly used.

As an additive of the tablet, powder, granule, capsule, pill, and troche according to the present invention, it is possible to use an excipient such as corn starch, potato starch, wheat starch, lactose, white sugar, glucose, fructose, D-mannitol, precipitated calcium carbonate, synthetic aluminum silicate, calcium monohydrogen phosphate, calcium sulfate, sodium chloride, sodium hydrogen carbonate, purified lanolin, microcrystalline cellulose, dextrin, sodium alginate, methyl cellulose, carboxymethyl cellulose sodium, kaolin, urea, colloidal silica gel, hydroxypropyl starch, hydroxypropyl methylcellulose (HPMC), HPMC 1928, HPMC 2208, HPMC 2906, HPMC 2910, propylene glycol, casein, calcium lactate, and Primojel; and a binder such as gelatin, arabic gum, ethanol, agar powder, cellulose acetate phthalate, carboxymethyl cellulose, carboxymethyl cellulose calcium, glucose, purified water, sodium caseinate, glycerin, stearic acid, carboxymethyl cellulose sodium, methylcellulose sodium, methylcellulose, microcrystalline cellulose, dextrin, hydroxycellulose, hydroxypropyl starch, hydroxymethyl cellulose, purified shellac, starch, hydroxypropyl cellulose, hydroxypropyl methyl cellulose, polyvinyl alcohol, and polyvinylpyrrolidone, and it is possible to use a disintegrant such as hydroxypropyl methyl cellulose, corn starch, agar powder, methyl cellulose, bentonite, hydroxypropyl starch, carboxymethyl cellulose sodium, sodium alginate, carboxymethyl cellulose calcium, calcium citrate, sodium lauryl sulfate, silicic anhydride, 1-hydroxypropyl cellulose, dextran, an ion exchange resin, polyvinyl acetate, formaldehyde-treated casein and gelatin, alginic acid, amylose, guar gum, sodium bicarbonate, polyvinylpyrrolidone, calcium phosphate, gelled starch, arabic gum, amylopectin, pectin, sodium polyphosphate, ethyl cellulose, white sugar, magnesium aluminum silicate, a D-sorbitol solution, and light anhydrous silicic acid; and a lubricant such as calcium stearate, magnesium stearate, stearic acid, hydrogenated vegetable oil, talc, lycopodium powder, kaolin, Vaseline, sodium stearate, cacao butter, sodium salicylate, magnesium salicylate, polyethylene glycol 4000, 6000, liquid paraffin, hydrogenated soybean oil (Lubriwax), aluminum stearate, zinc stearate, sodium lauryl sulfate, magnesium oxide, Macrogol, synthetic aluminum silicate, silicic anhydride, higher fatty acids, higher alcohols, silicone oil, paraffin oil, polyethylene glycol fatty acid ether, starch, sodium chloride, sodium acetate, sodium oleate, dl-leucine, and light anhydrous silicic acid.

As an additive for liquid formulation according to the present invention, it is possible to use water, diluted hydrochloric acid, diluted sulfuric acid, sodium citrate, sucrose monostearates, polyoxyethylene sorbitol fatty acid esters (Tween esters), polyoxyethylene monoalkyl ethers, lanolin ethers, lanolin esters, acetic acid, hydrochloric acid, aqueous ammonia, ammonium carbonate, potassium hydroxide, sodium hydroxide, prolamin, polyvinyl pyrrolidone, ethyl cellulose, carboxymethyl cellulose sodium, and the like.

In a syrup according to the present invention, a solution of sucrose, other sugars or sweeteners, and the like may be used, and a fragrance, a colorant, a preservative, a stabilizer, a suspending agent, an emulsifier, a thickener, and the like may be used, if necessary.

Purified water may be used for the emulsion according to the present invention, and an emulsifier, a preservative, a stabilizer, a fragrance, and the like may be used, if necessary.

In the suspending agent according to the present invention, a suspending agent such as acacia, tragacanth, methyl cellulose, carboxymethyl cellulose, carboxymethyl cellulose sodium, microcrystalline cellulose, sodium alginate, hydroxypropyl methyl cellulose, HPMC 1828, HPMC 2906, and HPMC 2910 may be used, and a surfactant, a preservative, a colorant, and a fragrance may be used, if necessary.

The injection according to the present invention may include: a solvent such as distilled water for injection, 0.9% sodium chloride injection, Ringer's injection, dextrose injection, dextrose+sodium chloride injection, PEG, lactated Ringer's injection, ethanol, propylene glycol, non-volatile oil-sesame oil, cottonseed oil, peanut oil, soybean oil, corn oil, ethyl oleate, isopropyl myristate, and benzoic acid benzene; a solubilizing aid such as sodium benzoate, sodium salicylate, sodium acetate, urea, urethane, monoethyl acetamide, butazolidin, propylene glycol, Tweens, nijungtinateamide, hexamine, and dimethylacetamide; a buffer such as a weak acid and a salt thereof (acetic acid and sodium acetate), a weak base and a salt thereof (ammonia and ammonium acetate), an organic compound, a protein, albumin, peptone, and gums; an isotonic agent such as sodium chloride; a stabilizer such as sodium bisulfite (NaHSO₃), carbon dioxide gas, sodium metabisulfite (Na₂S₂O₅), sodium sulfite (Na₂SO₃), nitrogen gas (N₂), and ethylenediaminetetraacetic acid; a sulfating agent such as 0.1% sodium bisulfide, sodium formaldehydesulfoxylate, thiourea, disodium ethylenediaminetetraacetate, and acetone sodium bisulfite; an analgesic such as benzyl alcohol, chlorobutanol, procaine hydrochloride, glucose, and calcium gluconate; and a suspending agent such as carboxymethyl cellulose sodium, sodium alginate, Tween 80, and aluminum monostearate.

In a suppository according to the present invention, it is possible to use a base such as cacao butter, lanolin, Witepsol, polyethylene glycol, glycerogelatin, methylcellulose, carboxymethyl cellulose, a mixture of stearic acid and oleic acid, Subanal, cottonseed oil, peanut oil, palm oil, cacao butter+cholesterol, lecithin, ranetwax, glycerol monostearate, Tween or Span, Imhausen, monolen (propylene glycol monostearate), glycerin, Adeps solidus, Buytyrum Tego-G, Cebes Pharma 16, hexalide base 95, Cotomar, Hydroxote SP, S-70-XXA, S-70-XX75(S-70-XX95), Hydrokote 25, Hydrokote 711, idropostal, Massa estrarium (A, AS, B, C, D, E, I, T), Massa-MF, Masupol, Masupol-15, Neosupostal-ene, Paramound-B, Suposhiro (OSI, OSIX, A, B, C, D, H, L), suppository base IV types (AB, B, A, BC, BBG, E, BGF, C, D, 299), Supostal (N, Es), Wecobee (W, R, S, M, Fs), and tegester triglyceride base (TG-95, MA, 57).

A solid formulation for oral administration includes a tablet, a pill, a powder, a granule, a capsule, and the like, and the solid formulation is prepared by mixing at least one excipient, for example, starch, calcium carbonate, sucrose or lactose, gelatin, and the like with the extract. Further, in addition to a simple excipient, lubricants such as magnesium stearate and talc are also used.

A liquid formulation for oral administration corresponds to a suspension, a liquid for internal use, an emulsion, a syrup, and the like, and the liquid formulation may include, in addition to water and liquid paraffin which are simple commonly used diluents, various excipients, for example, a wetting agent, a sweetener, a fragrance, a preservative, and the like. Examples of a formulation for parenteral administration include an aqueous sterile solution, a non-aqueous solvent, a suspension, an emulsion, a freeze-dried preparation, and a suppository. As the non-aqueous solvent and the suspension, it is possible to use propylene glycol, polyethylene glycol, a vegetable oil such as olive oil, an injectable ester such as ethyl oleate, and the like.

The pharmaceutical composition according to the present invention is administered in a pharmaceutically effective amount. In the present invention, “pharmaceutically effective amount” means an amount sufficient to treat diseases at a reasonable benefit/risk ratio applicable to medical treatment, and an effective dosage level may be determined according to factors including the type of disease of patients, the severity of disease, the activity of drugs, sensitivity to drugs, administration time, administration route, excretion rate, treatment period, and simultaneously used drugs, and other factors well known in the medical field. As a specific example, the pharmaceutical composition may be administered in an amount of 0.001 to 1000 mg/kg, 0.05 to 200 mg/kg or 0.1 to 100 mg/kg once or several times a day, and may be administered once as needed, not only in a weight-based dose but also in a flat-dose. A preferred dosage of the preparation of the present invention may be selected depending on the condition and body weight of a subject in need, the degree of a disease, the form of drug, the administration route, and the duration.

The pharmaceutical composition according to the present invention may be administered as an individual therapeutic agent or in combination with other therapeutic agents, may be administered sequentially or simultaneously with therapeutic agents in the related art, and may be administered in a single dose or multiple doses. It is important to administer the composition in a minimum amount that can obtain the maximum effect without any side effects, in consideration of all the aforementioned factors, and this amount may be easily determined by a person with ordinary skill in the art to which the present invention pertains.

The pharmaceutical composition of the present invention is determined by the type of drug that is an active ingredient, as well as various related factors such as the disease to be treated, the route of administration, the age, sex, and body weight of a patient, and the severity of the disease.

As used herein, the “subject in need” refers to a subject in need of treatment of a disease, and more specifically, may be a mammal such as a human or a non-human primate, a mouse, a rat, a dog, a cat, a horse, and a cow, but is not limited thereto.

The “administration” as used herein refers to the provision of a predetermined composition of the present invention to a subject in need thereof by any suitable method.

As used herein, the “prevention” refers to all actions that suppress or delay the onset of a target disease, and the “treatment” refers to all actions that ameliorate or beneficially change a target disease and the resulting metabolic abnormalities by administration of the pharmaceutical composition according to the present invention, and the “amelioration” refers to all actions that reduce a target disease and associated parameters, for example, the severity of symptoms, by administration of the composition according to the present invention.

As another aspect of the present invention, the present invention provides a pharmaceutical composition for preventing or treating cancer, including the above-described antibody-drug conjugate or a pharmaceutically acceptable salt thereof as an active ingredient.

As still another aspect of the present invention, the present invention provides a method for preventing or treating cancer, the method including: administering the above-described antibody-drug conjugate or a pharmaceutically acceptable salt thereof to a subject in need thereof.

All documents mentioned herein are incorporated herein by reference as if the contents thereof are described herein. When introducing elements of the present invention or the preferred embodiment(s) thereof, the articles “a”, “an”, “the” and “said” are intended to mean that there are one or more of the elements. The terms “comprising”, “including”, and “having” are intended to be inclusive and mean that there may be additional elements other than the listed elements. Although the present invention is described with respect to particular aspects or embodiments, it should not be construed as limiting the details of these aspects.

Hereinafter, preferred examples for helping the understanding of the present invention will be suggested. However, the following examples are provided only to more easily understand the present invention, and the contents of the present invention are not limited by the following examples.

### EXAMPLES

### Example 1. Preparation of Antibody-Drug Conjugate

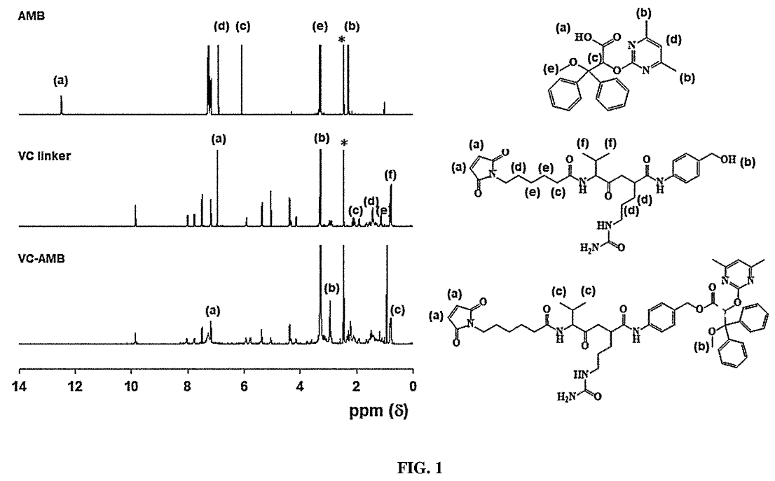

To develop an immune checkpoint inhibitor-based antibody-drug conjugate capable of exhibiting a high objective response rate, Fmoc-VC-AMB was prepared by stirring a peptide-based valine-citrulline (VC) linker (Fmoc-Val-Cit-PAB-OH, MedKoo) sensitive to an enzyme (Cathepsin B) overexpressed in a cancer microenvironment with an exosome secretion inhibitor ambrisentan (AMB) in the presence of 1-ethyl-3-(3-dimethylaminopropyl) carbodiimide·hydrochloride (EDC·HCl), and a 4-dimethylaminopyridine (DMAP) catalyst to form an ester bond. VC-AMB was prepared by removing Fmoc from the prepared Fmoc-VC-AMB in the presence of piperidine. After the prepared VC-AMB, AMB, and VC linker were dissolved using DMSO-d₆, the chemical structure of the prepared VC-AMB was identified by ¹H-NMR. The results are illustrated in FIG. 1.

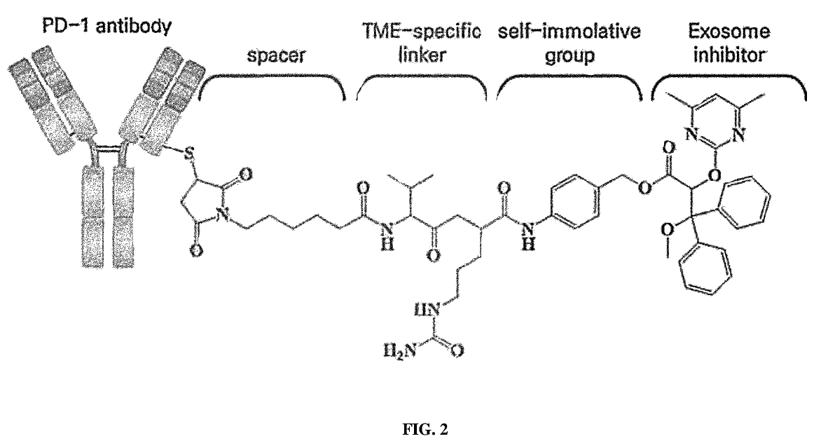

Mal-VC-AMB was prepared by chemically conjugating a spacer for antibody conjugation (6-maleimidohexanoic acid, Tokyo Chemical Industry) to the prepared VC-AMB. After an antibody was reduced by treating tris(2-carboxyethyl)phosphine (TCEP) with a pH 8.0 borate buffer at 25° C. for 30 minutes, VC-AMB was chemically conjugated to a PD-1 antibody (BioXCell, Clone: RMP1-14, Cat. No. BE0146) by adding a cold 20% acetnitrile solution to 1.1 eq Mal-VC-AMB of a free thiol group of an antibody determined through 5,5′dithiobis(2-nitrobenzoic acid (DTNB) at 4° C. Thereafter, the reaction was stopped by adding an excessive amount of cysteine thereto, and then an antibody-drug conjugate was obtained using a Zeba desalting column (Thermo). The structure of the prepared antibody-drug conjugate is illustrated in FIG. 2, and was named Ab-VC-AMB.

Absorbance of Ab, AMB and Ab-VC-AMB was measured using a UV-VIS spectrophotometer to confirm the drug-to-antibody ratio (DAR) of Ab-VC-AMB.

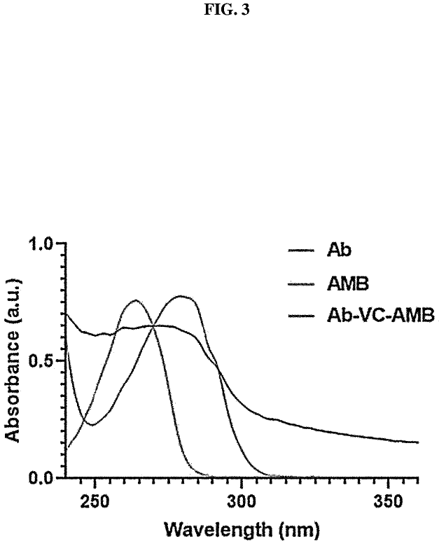

The absorbance measurement results are shown in FIG. 3, and the DAR of Ab-VC-AMB was calculated to be 3.53.

### Example 2. Cytotoxicity Evaluation of Ab-VC-AMB

In order to evaluate the cytotoxicity of Ab-VC-AMB prepared in Example 1, a melanoma cell line B16F10 (1×10⁴) was attached to a 96-well plate, and after 24 hours, and the cell line was treated with Ab, AMB, AMB+Ab, and Ab-VC-AMB at each concentration, respectively. After 24 hours, the number of living cells was calculated by MTT assay. For this, 5 mg/ml MTT reagent was diluted 1/10 in the medium, and then 200 μl was added to each well of the 96-well plate. After the cells were cultured in an incubator for 1.5 hours, the MTT reagent was removed, and 200 μl of DMSO was added to each well of the 96-well plate. After the cells were cultured at room temperature for 30 minutes, cell viability was calculated by confirming the absorbance at 570 nm.

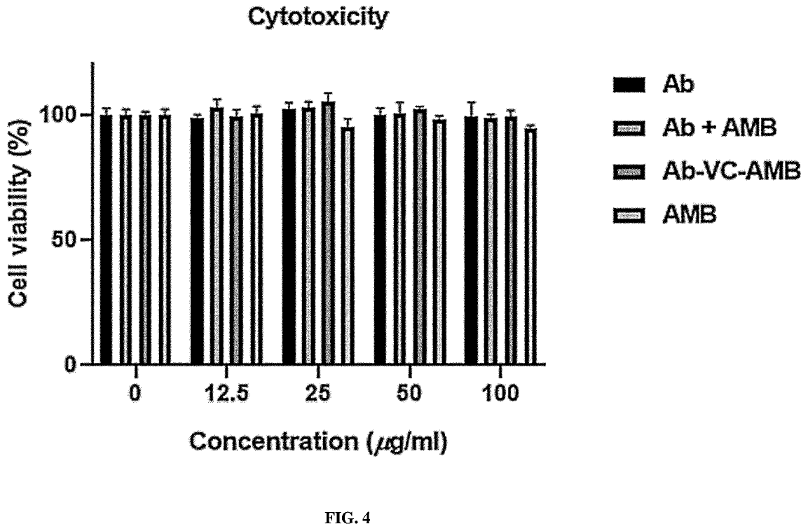

As a result, as illustrated in FIG. 4, it could be seen that Ab-VC-AMB had high biocompatibility because cytotoxicity was not observed in all groups. In addition, it was confirmed that AMB does not have a direct relationship with toxicity affecting cells because AMB acts as an endothelin receptor (ETA) antagonist.

### Example 3. Evaluation of Therapeutic Efficacy of Ab-VC-AMB in Disease Animal Model

In order to evaluate the therapeutic efficacy of Ab-VC-AMB prepared in Example 1 in a disease animal model, a cancer animal model was produced by subcutaneously inoculating B16F10 (1×10⁶ cells), which is a melanoma cell line, into mice and allowing tumors to grow for 10 days (Day 0). Thereafter, on days 1, 4, 7 and 10, Ab-VC-AMB, saline, an antibody (Ab) or AMB was injected intravenously into the cancer animal model (Ab 5 mg/kg, AMB 10 mg/kg, Ab-VC-AMB 5 mg/kg), and then the therapeutic efficacy was evaluated for 11 days.

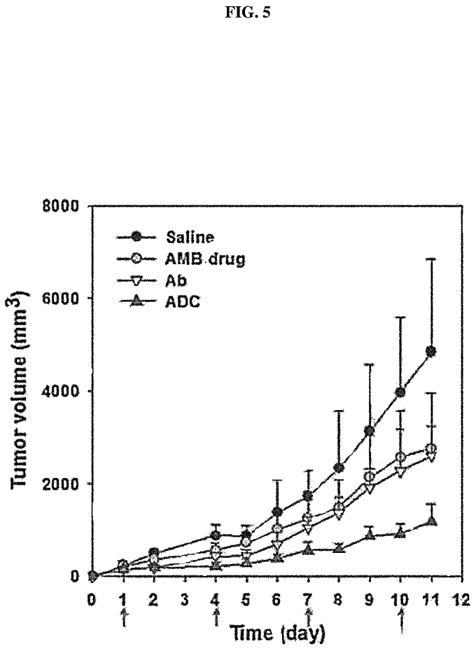

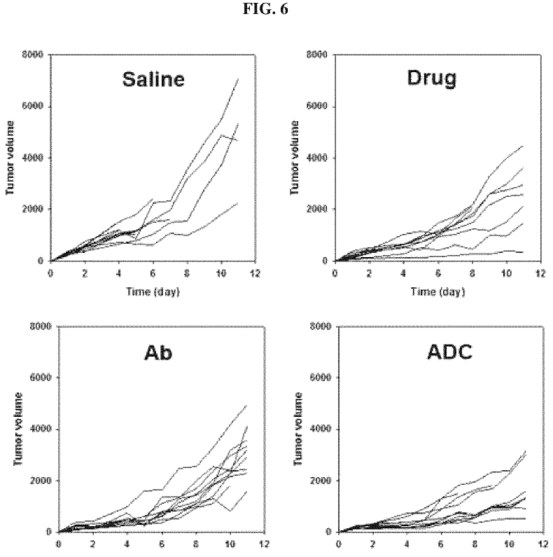

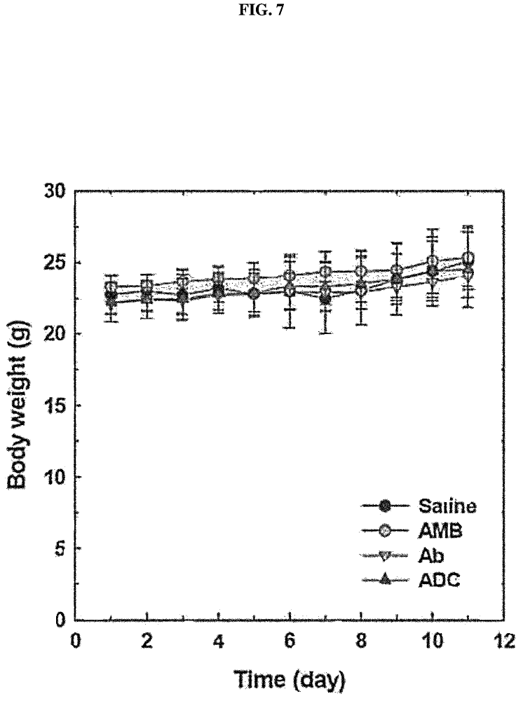

As a result, as illustrated in FIGS. 5 and 6, in the case of an Ab-VC-AMB experimental group, the cancer volume was at the level of 24% compared to the control to which saline was administered, cancer growth was remarkably suppressed, and the volume of cancer was about 45% even compared to the Ab control, showing high cancer therapeutic efficacy (FIGS. 5 and 6). In addition, each group showed similar levels of changes in body weight, indicating that Ab-VC-AMB does not show toxicity (FIG. 7).

A histopathological evaluation was performed by removing major organs and cancer tissue and staining the tissue by a hematoxylin and eosin (H&E) staining method. For this purpose, the major organs (liver, lungs, spleen, kidneys, and heart) and cancer tissue were removed, placed in a cassette, and fixed in a fixative solution. Then, after a paraffin block was prepared, a sample was prepared with a thickness of 6 μm. After the sample was stained with a Harris hematoxylin staining solution and an eosin-phloxine staining solution, the sample was observed through a slide scanner.

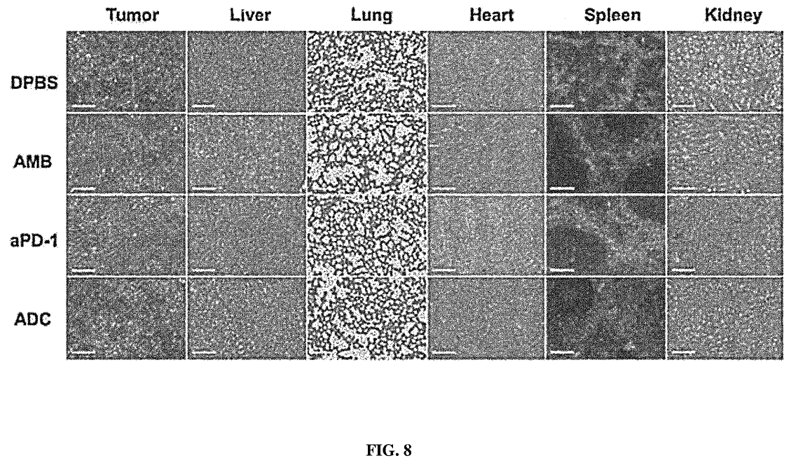

As a result, as illustrated in FIG. 8, the toxicity in the major organs was insignificant in all groups, and in the case of the Ab-VC-AMB experimental group, a large cell death area in the cancer tissue was observed compared to the control (FIG. 8).

The aforementioned results indicate that Ab-VC-AMB may effectively inhibit cancer growth.

### Example 4. Evaluation of Ability of Ab-VC-AMB to Inhibit Exosome Secretion in Disease Animal Model

To evaluate the ability of Ab-VC-AMB prepared in Example 1 to inhibit the secretion of exosomes, after the animal model for which the therapeutic efficacy evaluation was completed was sacrificed, plasma was isolated, and exosomes were isolated and extracted using an exosome isolation kit (Invitrogen total exosome isolation reagent). The amount of protein was measured by performing BCA analysis (bicinchoninic acid assay) on the isolated exosomes. Albumin standards diluted to various concentrations and 150 μl of an unknown sample whose protein amount was not known were put into a 96-well plate, respectively. 150 μl of a working reagent containing bicinchoninic acid from a BCA protein agonist kit (Pierce) was placed in each well containing each sample, and the resulting mixture was shaken and mixed well, and then incubated at 37° C. for 2 hours. Thereafter, the protein concentration of exosomes was calculated by measuring the absorbance at 562 nm using a microplate reader.

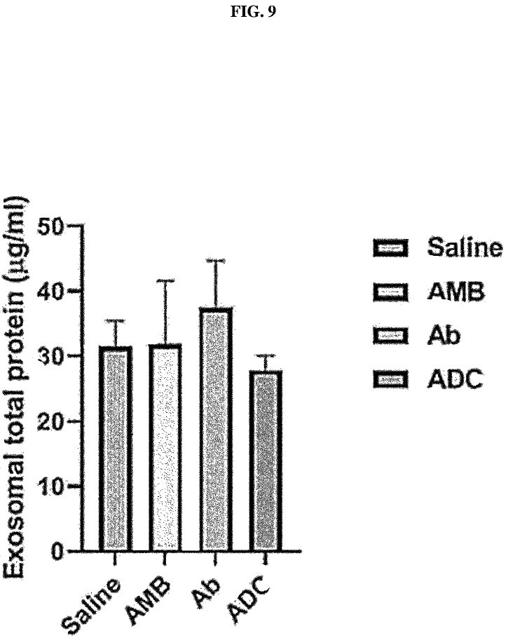

As a result, as illustrated in FIG. 9, the Ab-VC-AMB experimental group showed a statistically significant low amount of exosomal protein compared to the Ab control (FIG. 9). These results indicate that the total amount of protein contained in exosomes that suppress the immune response tends to be decrease.

In addition, PD-L1 on the surface of exosomes, which is a factor known to suppress the activity of T cells, was quantified through ELISA analysis. A 96-well plate was coated with 2 μg/ml PD-L1 antibody incubated at 4° C. for 16 hours at room temperature. After the plate was washed 3 times with phosphate-buffered saline with 0.05% Tween 20 (PBST), a blocking buffer was added thereto and the plate was incubated at room temperature for 2 hours. After the plate was washed 3 times with PBST, standards and samples using the serially diluted PD-L1 antibody were placed and left to stand at room temperature for 2 hours. After the plate was washed 3 times with PBST, a biotinylated PD-L1 detection antibody was added thereto, and the plate was incubated at room temperature for 2 hours. The plate was washed 3 times again, 40-fold-diluted streptavidin-conjugated peroxidase (Streptavidin-HRP) was added thereto, and the plate was incubated at room temperature for 20 minutes. After the plate was washed 3 times with PBST, a substrate solution in which H₂O₂ and tetramethylbenzidine were mixed at 1:1 was added to each well, and after the plate was incubated for 20 minutes, and the reaction was stopped by adding 2NH₂SO₄ thereto. Absorbance at 450 nm was measured using a microplate reader.

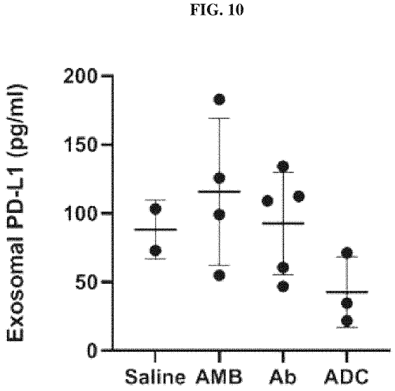

As a result, as illustrated in FIG. 10, the exosomal PD-L1 (PD-L1) level of exosomes showed a tendency to decrease remarkably in the Ab-VC-AMB experimental group compared to the other groups (FIG. 10). Such results mean that after the AMB drug was released from Ab-VC-AMB in response to the cancer microenvironment, the secretion of cancer exosomes was effectively inhibited, and are results supporting the high therapeutic efficacy of the Ab-VC-AMB experimental group in an anti-cancer treatment efficacy experiment through the disease animal model.

The above-described description of the present invention is provided for illustrative purposes, and those skilled in the art to which the present invention pertains will understand that the present invention can be easily modified into other specific forms without changing the technical spirit or essential features of the present invention. Therefore, it should be understood that the above-described embodiments are only exemplary in all aspects and are not restrictive.

### INDUSTRIAL APPLICABILITY

An antibody-drug conjugate according to the present invention is maintained, in normal tissue, in a form in which a drug is conjugated to an antibody, and releases the drug upon reaching a cancer microenvironment, thereby inhibiting the secretion of cancer exosomes that cause an immunosuppressive mechanism. Thus the antibody-drug conjugate exhibits high therapeutic efficacy and can remarkably increase the objective response rate to an immune checkpoint inhibitor, and thus has industrial applicability.

1. An antibody-drug conjugate (ADC) comprising: an antibody which is an immune checkpoint inhibitor; and an exosome secretion inhibitor conjugated to the antibody through a linker, or a pharmaceutically acceptable salt thereof.

2. The ADC or the pharmaceutically acceptable salt thereof of claim 1, wherein the antibody, which is an immune checkpoint inhibitor, is an antibody that specifically binds to programmed cell death 1 (PD-1) or PD-1 ligand 1 (PD-L1).

3. The ADC or the pharmaceutically acceptable salt thereof of claim 2, wherein the antibody that specifically binds to PD-1 is pembrolizumab, nivolumab or cemiplimab.

4. The ADC or the pharmaceutically acceptable salt thereof of claim 2, wherein the antibody that specifically binds to PD-L1 is atezolizumab, avelumab or durvalumab.

5. The ADC or the pharmaceutically acceptable salt thereof of claim 1, wherein the antibody, which is an immune checkpoint inhibitor, is an antibody that specifically binds to cytotoxic T-lymphocyte-associated protein 4 (CTLA4) or lymphocyte activation gene-3 (LAG-3).

6. The ADC or the pharmaceutically acceptable salt thereof of claim 5, wherein the antibody that specifically binds to CTLA4 is ipilimumab.

7. The ADC or the pharmaceutically acceptable salt thereof of claim 1, wherein the exosome secretion inhibitor is selected from the group consisting of Manumycin A, GW4869, cannabidiol and an endothelin receptor antagonist.

8. The ADC or the pharmaceutically acceptable salt thereof of claim 7, wherein the endothelin receptor antagonist is selected from the group consisting of ambrisentan, sulfisoxazole, BQ-123, BQ-788, zibotentan, sitaxentan, atrasentan, bosentan, macitentan, tezosentan and A192621.

9. The ADC or the pharmaceutically acceptable salt thereof of claim 1, wherein the linker is a cleavable linker which is cleaved in a cancer microenvironment, and the exosome secretion inhibitor is released by cleavage of the linker.

10. The ADC or the pharmaceutically acceptable salt thereof of claim 1, wherein the linker is a cleavable linker which is cleaved by a protease, and the exosome secretion inhibitor is released by cleavage of the linker.

11. The ADC or the pharmaceutically acceptable salt thereof of claim 10, wherein the protease is selected from the group consisting of Cathepsin B, Cathepsin K, a matrix metalloproteinase (MMP) and urokinase.

12. The ADC or the pharmaceutically acceptable salt thereof of claim 1, wherein the linker is a cleavable linker which is cleaved by acidity or reactive oxygen species of a cancer microenvironment, and the exosome secretion inhibitor is released by cleavage of the linker.

13. The ADC or the pharmaceutically acceptable salt thereof of claim 1, wherein the linker is a peptide linker.

14. The ADC or the pharmaceutically acceptable salt thereof of claim 13, wherein the linker is a cleavable linker which is cleaved by a protease.

15. The ADC or the pharmaceutically acceptable salt thereof of claim 13, wherein the peptide linker is a valine-citrulline linker.

16. A composition comprising the antibody-drug conjugate or the pharmaceutically acceptable salt thereof of claim 1.

17. A method for preventing or treating cancer, the method comprising: administering the antibody-drug conjugate or the pharmaceutically acceptable salt thereof of claim 1 to a subject in need thereof.

18. The method of claim 17, wherein the cancer is a cancer selected from the group consisting of lung cancer, gastric cancer, gliomas, liver cancer, melanoma, renal cancer, urothelial carcinoma, head and neck cancer, Merkel-cell carcinoma, prostate cancer, hematologic malignancy, breast cancer, colorectal cancer, colon cancer, rectal cancer, pancreatic cancer, brain cancer, ovarian cancer, bladder cancer, bronchial cancer, skin cancer, cervical cancer, endometrial cancer, esophageal cancer, thyroid cancer, bone cancer and a combination thereof.

19-20. (canceled)
